(19) United States
(12) Patent Application Publication
(10) Pub. No.: US 2014/0317767 A1
(43) Pub. Date: Oct. 23, 2014

(54) Persistent Inheritance of Hyperdominant Traits in a Perennial Lineage

(71) Applicant: William Douglas Meadow, JR., Jacksonville, FL (US)

(72) Inventor: William Douglas Meadow, JR., Jacksonville, FL (US)

(21) Appl. No.: 14/206,780

(22) Filed: Mar. 12, 2014

Related U.S. Application Data
(60) Provisional application No. 61/786,275, filed on Mar. 14, 2013.

(51) Int. Cl. A01H 1/04; A01K 67/027

(52) U.S. Cl. CPC A01H 1/04; A01K 67/027
USPC 800/21; 800/260

(57) ABSTRACT

The present invention provides a perennial lineage engendered with hyperdominant traits that express consistent penetrance in all descendants of a founder organism. A genetic construct containing one or more genetic elements encoding a self-regulating feedback loop generates a regulatory RNA, polypeptide, or other gene product at or below a trigger level of concentration in a zygote and indicates with respect to the concentration level whether one or two copies of a genetic construct conferring a hyperdominant trait exist in said zygote.

### CROSS REFERENCE TO RELATED APPLICATIONS

The present application claims priority to provisional patent application Ser. No. 61/786,275, filed Mar. 14, 2013 and entitled, “Persistent Inheritance of Hyperdominant Traits in a Perennial Lineage”, the contents of which are relied upon and incorporated by reference.

### FIELD OF INVENTION

The present invention relates to a novel mode of hyperdominant inheritance and the persistent expression of traits across generations without dilution. In some embodiments a process may occur by which a hyperdominant trait can be recreated across successive generations with fidelity in a manner materially distinct from the presently understood patterns of genetics and the inheritance of Mendelian dominant genes in scenarios involving abundant wild-types and a monophyletic lineage.

### BACKGROUND OF THE INVENTION

Nature's existing processes for the generation and vertical transmission of genetic adaptations leave much to be desired in regard to ensuring the proliferation of a new adaptation or trait across a large population, or in a large proportion of an ancestral organism's eventual descendants. In the case of plant and animal taxa, particularly those which use sexual reproduction to recombine haploid genomes into new diploid organisms, a trait exhibiting Mendelian dominance may soon fade from a lineage of ancestors to descendants. For instance, an organism homozygous for a dominant trait produces heterozygous offspring following a cross with a wild-type organism to yield the first filial generation. These heterozygous offspring may cross with wild-type organisms as well, to yield a second filial generation which is expected to have half its organisms bearing a heterozygous dominant trait while the other half are themselves wild-type organisms indistinguishable from those not descended from the founder homozygous for the dominant trait. This scenario, as described, assumes that the trait exhibits consistent penetrance and follows a Mendelian dominant pattern of inheritance.

The result which follows from this naturally occurring pattern of inheritance is that even a so-called dominant trait may fade within two generations of a progenitor, or founder organism which originates a new, beneficial trait or even is homozygous for it. Otherwise stated, a beneficial adaptation may be diluted out to a low relative preponderance among a large set of reproductively compatible organisms. If the selective advantage happens to be slight, it may be many generations before the allele becomes fixed in a syngameon, species, or even a regional population. The biological process upon which this pattern rests is that fertilization combines, with the exclusion of circumstances involving aneuploidy, equal nuclear DNA contributions from each haploid parental genome. Once an adaptation is diluted by half in a heterozygous genome, the beneficial contribution of one parent's trait is not weighed against a less adaptive contribution from the other parent's haploid genome not encoding the trait. Essentially, the feature of randomly assorting either parent's haploid genome contribution into gametes with respect to a particular locus means that potentially half the progeny of a heterozygous organism and a wild-type organism may lack a specific contribution, adaptive with respect to natural selection, of the heterozygous organism's parent. A pattern of Mendelian dominance, or even a gene which exhibits complete penetrance, cannot mitigate this fact if the core barrier to the proliferation of the trait within the lineage of the founder's descendants constitutes an initially low prevalence of the trait, or even its absence, from the population at large with which the founder's descendants interbreed. To describe the relative preponderance of descendants of the founder organism which lack the founder's adaptive trait, the lineage proceeding from the founder originating the new adaptation can be said to be not perennial.

Exceptions to Mendelian inheritance exist as naturally occurring instances within plant and animal taxa. For instance, observations of the phenomenon known among plants as apomixis teach that a parental organism can ensure that its traits are passed to all descendants in a perennial manner, forgoing the contingency of a de novo loss of function mutation that may eliminate the trait. In parthenogenesis, the maternal genome is passed down via either haploid parthenogenesis or diploid parthenogenesis such that no paternal gametes may interfere or dilute out the maternal genetic contribution. Furthermore, the conceptually parallel natural phenomenon of androgenesis involves fertilization of female gametes by male gametes which replace the maternal nuclear genetic material to yield progeny that are paternal clones with respect to their nuclear genomes, though cytoplasmic DNA such as that within chloroplasts and mitochondria may remain as a maternal genetic contribution. In each case, a founder organism originating a new, adaptive trait conferring a selective advantage may be a progenitor of a perennial lineage in a sex-specific manner. A parthenogenetic female parent can found a perennial lineage of females bearing a particular trait given consistent penetrance and the continuation of parthenogenetic reproductive behavior over the ensuing generations. Through androgenesis, a male parent can found a perennial lineage of males whose nuclear genomes cause them to bear a particular trait, given consistent penetrance and the continuation of androgenetic reproductive behavior over the ensuing generations.

In the process of meiosis, segregation distortion exists such that a given genetic locus may be present in more than 50% of mature gametes formed by a heterozygous organism exhibiting this phenomenon. For instance, the segregation distorter (Sd) locus in Drosophila melanogaster exhibits the phenomenon that male gametes lacking this allele very often fail to become viable sperm cells in a heterozygous male fruit fly. Research by Selena Gell and Robert Reenan indicates a responder (Rsp) locus whose copy number correlates to sensitivity to the Sd locus on chromosome 2 of Drosophila. While this natural process can strongly favor the presence of a particular Sd locus and perhaps neighboring linked loci in the mature, functional gametes of an Sd heterozygote fruit fly, this mechanism does not teach a way to ensure a stable, perennial lineage of homozygotes after repeated crosses with wild-type organisms. Rather, it only teaches a means by which heterozygotes may preferentially select a given locus to be in their mature gametes. Notably, some traits exhibit different phenotypes under the heterozygous and homozygous conditions, and a heterozygous genotype can be prone to a lower penetrance of its corresponding trait when compared to a homozygous genotype. If only one copy of the Sd locus is provided by a paternal gamete to a wild-type female gamete, then any daughter organisms from this cross will produce gametes by the typical Mendelian segregation characteristic of a heterozygote. A homozygous Sd male or female in a parental generation may thus yield all heterozygote offspring in the first filial F1 generation following a cross with wild-types. In a second filial F2 generation following a cross with wild-types, those F2 progeny derived from F1 heterozygous males may be approximately 99% Sd heterozygous, while those F2 progeny derived from F1 heterozygous females may be approximately 50% Sd heterozygous and 50% wild-type. Following successive generations and the decreasing percentage of total progeny in an exclusively male direct lineage, the Sd locus may further dilute out its abundance in a population with a preponderance of wild-type organisms. This natural process is restricted to activity that takes place during meiosis and the generation of gametes. It does not teach how processes in a zygote may be used toward the end of a perennial lineage.

These naturally occurring phenomena among plant and animal taxa does not instruct how a perennial lineage, namely, one bearing a particular trait generation after generation without population-caused dilution or diminishing penetrance, can emerge from a founder organism with an adaptive trait under more standard conditions. Considering a scenario which involves sexual reproduction, haploid genetic contributions by each of two diploid parents, and either a limited presence or the absence of a new, selectively advantageous adaptation among a population, there is not a naturally occurring method for a founder organism bearing such an adaption to ensure that this adaptation is passed to all progeny despite extensive crossing with population members lacking the adaptation. A scenario in which lacking the adaptation prevents survival or reproduction does not create a novel method of inheritance, since it is presumed that the adaptation is already scarce or non-existent among a population which can survive and reproduce independently of a new, selectively advantageous adaptation which has yet to proliferate in the population. Expressed another way, for a founder organism to pass a trait that confers a less than absolute selective advantage on to all its progeny in a monophyletic lineage, particularly using a system of sexual reproduction, haploid genetic contributions by each of two diploid parents, with this trait being absent or minimally prevalent in the population with which the founder's descendants interbreed, may be a nontrivial result with no obvious naturally occurring biological underpinning. In brief, the hyperdominant pattern of inheritance following sexual reproduction and haploid nuclear contributions by each parent is not present in apomixis, androgenesis, parthenogenesis, or single-sex meiotic segregation distortion as a natural mode of inheritance. It may be desirable to develop methods and apparatus that will allow for persistent inheritance of hyperdominant traits in a perennial lineage.

### SUMMARY DESCRIPTION OF INVENTION

Accordingly, the present invention provides a perennial lineage engendered with hyperdominant traits that express consistent penetrance in all descendants of a founder organism. A genetic construct containing one or more genetic elements encoding a self-regulating feedback loop generates a regulatory RNA, polypeptide, or other gene product at or below a trigger level of concentration in a zygote and indicates with respect to the concentration level whether one or two copies of a genetic construct conferring a hyperdominant trait exist in said zygote.

A hyperdominant trait is one which is not subject to a decrease in prevalence among the descendants of a founder organism owing to repeated reproductive crosses with wild-type organisms lacking the trait. The hyperdominant pattern of inheritance is thus resilient to environmental factors, such as a trait conferring a less than absolute selective advantage, or the previous absence or low prevalence of the trait among members of the population with which the founder organism's descendants interbreed. In particular, this resilience is realized relative to a pattern of Mendelian so-called complete dominant inheritance where a homozygous dominant organism may have descendants lacking the trait as early as the second filial generation. After an arbitrarily large number of generations in which the descendants of a founder organism bearing a hyperdominant trait interbreed with wild-type organisms from an arbitrarily large population, the proportion of the founder organism's descendants bearing the hyperdominant trait does not exhibit the exponential decay of halving with each successive generation, but rather continues to remain at unity. This result may not be a natural mode of inheritance and this result may introduce the meaning of a hyperdominant trait related to the inventive art described herein.

A perennial lineage begins with a founder organism bearing a hyperdominant trait, where the founder's parents did not bear the hyperdominant trait, or else the organism may not be of the founding generation of the lineage of organisms bearing this trait. In addition to the founder organism, the entirety of the founder organism's eventual progeny, an arbitrary number of generations removed, constitute the remainder of the perennial lineage. The contingency of a de novo loss of function mutation that might eliminate the trait is addressed as an unavoidable consequence of the biological fact of a nonzero background rate of mutation. It is otherwise presumed that all members of the perennial lineage bear the hyperdominant trait, as a consequence of the patterns and mechanisms of inheritance commensurate with the invention's novel processes.

Hyperdominance can be divided into three overall classes based upon the genomic location of the genetic material that is responsible for the hyperdominant trait.

In the first class, an episomal hyperdominant trait is encoded by a genetic construct which is not part of the usual complement of chromosomes comprising the organism's genome, or of the complement of chromosomes comprising the genomes of other members of the organism's species. Instead, episomal hyperdominance relies on the introduction of a foreign, artificial, or recombinant chromosome that is not part of the natural, pre-existing genetic heritage of the organism or the organism's species. Episomal hyperdominance may be further divided into subclasses based upon the manner of interaction between multiple episomal hyperdominant traits to produce a novel pattern of trait inheritance within a perennial lineage.

In the second class, an autosomal hyperdominant trait is encoded by a genetic construct which is a subordinate part of the usual complement of chromosomes comprising the organism's genome, or of the complement of chromosomes comprising the genomes of other members of the organism's species. Autosomal hyperdominance relies on the modification of existing autosomal chromosomes or on the introduction of modified analogs to existing autosomal chromosomes. Autosomal hyperdominance may be further divided into types based upon the manner of interactions between homologous pairs of autosomal chromosomes which give rise to the novel emergent pattern of hyperdominant trait inheritance within a perennial lineage.

In the third class, allosomal hyperdominance, the sex chromosomes of an organism interact in a manner akin to that of autosomal or episomal hyperdominance so as to allow traits encoded by parts of a sex chromosome to also exhibit a hyperdominant pattern of inheritance within a perennial lineage. Allosomal hyperdominance can also contribute to stabilizing selection toward a given sex ratio within a population depending on operational parameters for allosomal chromosome interactions used in conjunction with other relevant factors such as the existing sex ratio as well as survival and reproduction rate parameters.

There may be numerous methods by which one can implement hyperdominant techniques of inheritance. The core steps involved in the chromosomal interaction which provides for the characteristic pattern of inheritance responsible for a hyperdominant trait's perennial persistence across generations may be as follows. If both contributing gametes to a zygote carry the genetic construct associated with hyperdominance, known as the hyperdominant region, the mechanism of the invention is not needed to ensure that the organism derived from the zygote bears the hyperdominant trait. In any case in which a genetic construct yielding a hyperdominant trait would otherwise make only a hemizygous or heterozygous contribution to an organism's genome at the stage of a new zygote, a self-regulating feedback loop ensures that an appropriate genetic replication takes place such that the organism has two copies of the genetic region associated with the hyperdominant trait. In special cases, only a portion of the genetic region associated with hyperdominant trait may be replicated. Along with consistent penetrance of the genetic construct to yield the hyperdominant trait in the organism derived from the zygote, the two copies of the genetic region associated with the hyperdominant trait also ensure its presence in the haploid gametes produced by the organism by means of the sorting of the nuclear genome during meiosis. In special cases, the feedback loop may act prior to or during gametogenesis to ensure that all mature gametes bear the hyperdominant region. In this scenario, the contingency of non-disjunction is considered an unavoidable consequence of biologically imperfect meiosis. This, like the aforementioned nonzero background rate of new genetic mutations, may not be part of the invention itself. It is otherwise presumed that all gametes formed by organisms within a perennial lineage have the same undiluted potential to continue the hyperdominant pattern of trait inheritance within the perennial lineage.

Each of the three classes of hyperdominance: episomal, autosomal, and allosomal, has particular attributes and processes associated with its particular mode of specialization within the subject area of the invention. A self-regulating feedback loop may be provided by genetic elements for one or more of a regulatory RNA, polypeptide, or other gene product which undergo transcription from DNA into RNA and may be translated from RNA into a polypeptide to reflect how many copies of the genetic elements encoding the self-regulating feedback loop in this prescribed manner exist within a zygote. Downstream signal processing may incorporate transcription factors and DNA-binding proteins to accommodate the zygote's reaction to the copy number ascertained by the feedback loop.

In the class of episomal hyperdominance, the genetic construct contains one or more genetic elements encoding a feedback loop. One copy of the episome generates a regulatory RNA, polypeptide, or other gene product which then exists in a zygote at a certain trigger level which causes the episome to replicate independently from other chromosomes in the genome of a zygote. In the event that both gametes combining to form a zygote each bear a copy of the episomal genetic construct, the resultant zygote has two copies of the episome to begin with. Critical to the operation of the feedback loop may be a gene product expression level corresponding to one copy of the episome does initiate autonomous episome replication independent of the rest of the genome and prior to the first division of the zygote, while a gene product expression level corresponding to that which ensues from the presence of two copies of the episome does not initiate episome replication other than that which is coordinated with the rest of the organism's genome in conjunction with mitosis of the zygote and its derived organism.

A genome which otherwise may have been hemizygous with respect to the episomal genetic construct then has two resultant copies of the episomal genetic construct responsible for the hyperdominant trait. Continuing in the perennial lineage, gametes formed by the organism derived from the zygote may carry one copy of the episomal genetic construct responsible for the hyperdominant trait as the diploid genome contains two such episomes while the haploid genome contains one. Even after an arbitrary number of crosses with wild-type organisms lacking the hyperdominant trait and its episomal genetic construct source, over an arbitrary number of generations, all descendants from a founder organism within the perennial lineage can continue to produce the hyperdominant trait inheritance taught by the invention. Two or more episomal genetic constructs which are capable of recognizing another's presence through a common feedback loop of gene product expression in a zygote, thus not needing to replicate to ensure diploidy of the episomal genetic construct, can be said to be within the same subclass of the class of hyperdominance known as episomal hyperdominance.

It may be possible for an assortment of episomal genetic constructs which correspond to variations on a single hyperdominant trait to provide genetic diversity among a population where all descendants of perennial lineage founder organisms express a common hyperdominant trait, owing to the consistent penetrance of the episomal genetic construct. The existence of some variation within a subclass of mutually recognizable episomal genetic constructs which coordinate to yield a common hyperdominant trait or common set of traits allows for genetic variation to persist in a population among the organisms who are part of one or more perennial lineages associated with a single hyperdominant trait.

The second class of hyperdominance is autosomal hyperdominance. In this case, the subclasses of mutually compatible episomal genetic constructs corresponding to a common hyperdominant trait may have a near analog in the behavior of chromosome pairs involved in autosomal hyperdominance. The homologous chromosome pair that exists for each autosome within the genome of a diploid organism provides the frame of reference from which a genetic region on an autosome corresponding to a hyperdominant trait can convert particular loci of the autosome from a state of being heterozygous within a new zygote to being homozygous within a new zygote before any mitotic divisions take place. Rather than the hemizygous to homozygous transition seen in the correcting mechanism which occurs in a zygote to preserve the perennial lineage and the hyperdominant trait corresponding to a subclass of episomal genetic construct, a heterozygous to homozygous transition is used. This shift in terminology reflects the difference between the scenario involving an episome with no homolog, the hemizygous condition and the scenario involving an autosome which has a homolog that largely resembles said autosome in structure, size, and placement of genetic loci. The homologous autosomal chromosome lacking the genetic locus corresponding to the introduced hyperdominant trait is already in place. When the heterozygous to homozygous transition takes place in a zygote, this removes the means by which the organism derived from the zygote may otherwise be able to eventually produce gametes lacking, on a particular autosome, the genetic region responsible for the hyperdominant trait. The removal of this means by which heterozygotes for dominant traits are wont to fail to pass on the trait, thus ending a lineage of organisms bearing the trait, results in the integrity of a perennial lineage by means of the hyperdominant mode of inheritance. Though the particular autosome chosen for autosomal hyperdominance depends on the hyperdominant trait in question, the mechanism for hyperdominance in a genetic region with respect to a particular autosome and selected loci along the autosome can be subdivided into several types: 1, 2a, and 2b. It may be possible that distinct types of autosomal hyperdominance may be predicated upon diverse means of a heterozygous to homozygous transition.

In type 1 autosomal hyperdominance, the replacement of existing genetic material on a given autosome among a population is favored over preserving existing genetic diversity on the given autosome which is to correspond to the hyperdominant trait. In a scenario where a gamete bearing an autosome responsible for a hyperdominant trait, henceforth a hyperdominant autosome, meets a gamete bearing the wild-type, non hyperdominant version of the homologous autosome, a heterozygote initially results. The genetic locus corresponding to the introduced hyperdominant trait exists in a single copy in such a scenario; it exists in two copies if both contributing gametes already bear a hyperdominant autosome in that homologous chromosome pair. By means of genetic elements on the hyperdominant autosome which encode a self-regulating feedback loop, the zygote generates one of two biochemical states which determine if genetic replication is necessary or not. Specifically, these genetic elements of the hyperdominant autosome induce the expression of a regulatory RNA, polypeptide, or other gene product in a way that enables self-regulation of the genetic replication process. If multiple hyperdominant regions exist on the same autosome as part of engendering distinct hyperdominant traits, each may use its own independent feedback loops. This ensures the perennial inheritance of both traits with fidelity, even in zygotes resulting from parents that each had one trait and not the other, as part of two perennial lineages in the same population combining their traits which are associated on a common autosome. This can be considered akin to the manner in which subclasses of episomal hyperdominant constructs maintain independent feedback loops, where a particular episomal construct subclass corresponds to a given hyperdominant region on an autosome. In the event that two hyperdominant autosomes of a homologous pair exist in the zygote, no genetic replication is needed to ensure that the organism derived from the zygote both bears the hyperdominant trait and continues the perennial lineage with an undiminished genetic inheritance regarding the hyperdominant trait. In the event that only one hyperdominant autosome is present in the zygote with respect to a particular homologous chromosome pair, the regulatory RNA, polypeptide, or other gene product then exists in the zygote at an aforementioned trigger level using the same principle of the feedback loop described within the class of episomal hyperdominance. Within the class of autosomal hyperdominance, type 1 is predicated upon the most complete heterozygous to homozygous transition relative to the latter types, 2a and 2b. In particular, the feedback loop's detection of a single copy of the hyperdominant autosome initiates degradation of the wild-type autosome and the specific replication of the hyperdominant autosome independently of the remainder of the genome. This takes place within the zygote such that the organism derived from the zygote is homozygous for all genetic loci on the given autosome, necessarily including the regions of the autosome which corresponds to the hyperdominant trait.

In both variants of type 2 autosomal hyperdominance, genetic elements of the hyperdominant autosome may be involved in the heterozygous to homozygous transition in the corresponding homologous chromosome, though at least some genetic loci of the non hyperdominant version of the homologous autosome remain intact. This allows for some genetic diversity in the form of heterozygosity at loci not directly involved with the generation of the hyperdominant trait as the zygote develops as an organism. This approach takes into consideration the biological fact that genes whose products are involved in a wide array of disparate functions may all be located on a common autosomal chromosome within an organism's genome.

In type 2a autosomal hyperdominance, the genetic region with respect to a particular autosome which contains genetic elements encoding a self-regulating feedback loop as well as the hyperdominant trait itself is considered separately from the remainder of the genetic loci on the autosome. Collectively, these may be considered the hyperdominant region of the autosome and represent less than the entirety of the autosome. In the event of type 2a autosomal hyperdominance resulting in an expression level of the regulatory RNA, polypeptide, or other gene product corresponding to a single hyperdominant region among the two homologous autosomes in the genome of a zygote, the heterozygous to homozygous transition appropriate for type 2a autosomal hyperdominance is initiated. Specifically, the process of homologous recombination and homologous chromosome DNA template repair via dsDNA breaks is used to preferentially replace the region of the wild-type autosome corresponding to the hyperdominant region found only on the hyperdominant autosome. Any genetic material which is distributed to the successive generations of a perennial lineage by virtue of proximity to the genetic elements encoding the self-regulating feedback loop as well as the hyperdominant trait itself, despite being not necessary for the hyperdominant trait's consistent penetrance, can exhibit gene linkage to the hyperdominant region of the autosome provided that the observed crossover frequency implies more than the 50% association seen in independent assortment yet less than the 100% linkage of the hyperdominant region proper. Type 2a thus allows a hyperdominant trait to persist across a perennial lineage while also preserving genetic diversity elsewhere on the autosome.

In type 2b autosomal hyperdominance, the genetic region with respect to a particular autosome which contains genetic elements encoding a self-regulating feedback loop as well as the hyperdominant trait itself may be considered separately from the remainder of the genetic loci on the autosome. As in type 2a autosomal hyperdominance, these genetic elements responsible for the self-regulating feedback loop and the hyperdominant trait itself are considered together the hyperdominant region of the autosome. In type 2b autosomal hyperdominance, the trait involved may exhibit Mendelian complete dominance such that the phenotypes of homozygotes and heterozygotes for the genetic region responsible for the hyperdominant trait are indistinguishable. In the event of type 2b autosomal hyperdominance resulting in an expression level of the regulatory RNA, polypeptide, or other gene product corresponding to a single hyperdominant region among the two homologous autosomes in the genome of a zygote, the heterozygous to homozygous transition appropriate for type 2b autosomal hyperdominance may be initiated. Since copying over the genetic region within the hyperdominant region responsible for the hyperdominant trait may produce no noticeable change in phenotype in the case of complete dominance, only the genetic elements encoding the self-regulating feedback loop may be copied over within the hyperdominant region. Specifically, the process of homologous recombination and homologous chromosome DNA template repair via dsDNA breaks may be used to preferentially copy these genetic elements from the type 2b hyperdominant autosome to the wild-type autosome. The second copy of the genetic elements of the self-regulating feedback loop allow the zygote to cease the heterozygous to homozygous transition when the process is completed for all relevant genetic loci on the homologous autosome pair.

In type 2b autosomal hyperdominance, while only a single copy of a genetic region responsible for the hyperdominant trait may be needed to produce the observable phenotype, other genetic loci on the autosome may be made homozygous as part of the genetic heritage of the perennial lineage. Specifically, at least some genetic loci outside the hyperdominant region that corresponds to the self-regulating feedback loop and the hyperdominant trait undergo homologous recombination and homologous chromosome DNA template repair via dsDNA breaks such that said genetic loci from the hyperdominant autosome are reflected on the formerly wild-type autosome. As mentioned in the discussion of type 2a autosomal hyperdominance, genetic material in proximity to the hyperdominant region on an autosome may be distributed to successive generations in a perennial lineage through gene linkage. In type 2a autosomal hyperdominance, this is not a directed process but rather the consequence of biological variation in the precise location of the endpoints of homologous recombination and dsDNA breaks in the homologous chromosome DNA template repair process along the autosome. In type 2b autosomal hyperdominance, key differences may include that the genetic material copied over consists of some, but not all of the hyperdominant region (namely, the genetic elements for the self-regulating feedback loop in exclusion of the genetic elements engendering the hyperdominant trait) and that the mechanism may provide for the directed heterozygous to homozygous transition of genetic loci outside of the hyperdominant region. Owing to the variable locations where dsDNA breaks and homologous recombination endpoints occur in each instance of this process, such genetic loci are not considered part of the hyperdominant trait (or set of hyperdominant traits) whose inheritance is made certain, but rather experience a higher rate of being passed along with the hyperdominant region across generations than genetic loci whose inheritance is not favored by this effective process of induced gene linkage. If the inheritance of these additional genetic loci outside the hyperdominant region were made certain through the selection of parameters controlling the heterozygous to homozygous conversion process, then their inheritance may properly be described as type 2a autosomal hyperdominance over an expanded hyperdominant region. As such, to describe type 2b autosomal hyperdominance as a distinct process from type 2a with respect to deliberate heterozygous to homozygous conversion of genetic loci outside the hyperdominant region, it may be desirable that this conversion is directed to occur in excess of that which is observed from incidental conversion of genetic loci on the edge of the smallest genetic region which must be reliably copied over for successful type 2a autosomal hyperdominance. In either case, the linkage may be stronger with greater proximity to the copied over region and progressively trend toward no gene linkage with increasing distance from the endpoints of dsDNA breaks, homologous recombination, and homologous chromosome DNA template repair.

Type 2b autosomal hyperdominance allows for heterozygosity in the genetic region corresponding to the complete dominant hyperdominant trait (indistinguishable heterozygotes and homozygotes) while also enforcing homozygy in at least some other genetic loci on the autosome. In type 1 and type 2a hyperdominance, it is possible for the hyperdominant trait to not be completely dominant. This may be irrelevant to those mechanisms since heterozygotes with one copy of the hyperdominant region may not be part of the inheritance patterns of type 1 and type 2a hyperdominance, such that their different phenotype in the case of incomplete dominance never develops. However, type 2b autosomal hyperdominance can be most effectively used with a complete dominant trait, such that the phenotype is constant even in the heterozygotes for the genetic region corresponding to the complete dominant hyperdominant trait. Type 2b autosomal hyperdominance with an incomplete dominant trait could still be considered as giving rise to a perennial lineage in the case that the heterozygotes for the genetic region corresponding to the hyperdominant trait still produced a phenotype distinct from that of the wild-types in the population at large, with the added expectation that the mechanism may lead to homozygotes for the genetic region corresponding to the hyperdominant trait after enough permeation of the type 2b hyperdominant autosome in the population.

Further usage of the hyperdominant feedback loop may take place in the generation of viable gametes in a type 2b autosomal hyperdominant heterozygote to ensure that the hyperdominant region may be on all copies of the autosome within mature gametes. Also, the hyperdominant region may be copied over prior to meiosis, guided by hyperdominant feedback loop input, to accomplish the same end. Meiotic product selection may also be employed as part of type 2b. In a manner that may be different from type 2a hyperdominance, the hyperdominant region may be copied over along with the other genetic loci where homozygosity is enforced at the zygote stage such that eventual type 2b autosomal hyperdominant gametes all bear the hyperdominant region. Any of these methods may be utilized in conjunction with applying type 2b autosomal hyperdominance. A distinction between type 2a and type 2b autosomal hyperdominance may be that type 2a autosomal hyperdominance may not specifically target heterozygous to homozygous beyond the core hyperdominant region of the autosome required for the hyperdominant trait and the feedback loop, aside from incidental linkage effects from proximity. Type 2b autosomal hyperdominance may act at additional genetic loci on the autosome beyond that of the portion of the hyperdominant region responsible for the hyperdominant trait. Specifically, it may constitutively enforce the heterozygous to homozygous conversion at the portion of the hyperdominant region corresponding to the feedback loop, while also having the capability to direct heterozygous to homozygous conversion at genetic loci outside of the hyperdominant region responsible for the hyperdominant trait in question.

In allosomal hyperdominance, the self-regulating feedback loop described in episomal and autosomal hyperdominance may produce a similar mechanism of inheritance. Specifically, for genes located on the X and Y chromosomes, or on the Z and W chromosomes, a hyperdominant region may enforce the heterozygote to homozygote transition as described in autosomal hyperdominance types 2a and 2b such that a genetic locus common to both sex chromosomes can exhibit partial allosomal hyperdominance. The mechanisms involved may not be materially different from those described previously for autosomal hyperdominance 2a and 2b, but rather may take place with the additional consideration that even the homologous regions of paired genetic loci on different sex chromosomes may not be classified as autosomes. Since, in some embodiments not all of the allosome may not be copied over in this transition, this category of allosomal hyperdominance is known as partial allosomal hyperdominance. Partial allosomal hyperdominance can result in a perennial lineage with a hyperdominant trait corresponding to genetic loci on an allosome. Total allosomal hyperdominance may occur in some embodiments of the inventive art herein.

Under total allosomal hyperdominance, a pattern in which a hyperdominant Xh chromosome exists alongside a wild-type Xw chromosome and the Y chromosome in a population, a pattern more similar to type 1 autosomal hyperdominance may take place. In a zygote formed by Xw Y and Xw Xw parents, the four possible outcomes are: 2 Xw Y:2 Xw Xw. This may correspond to the existing pattern observed in the absence of total allosomal hyperdominance. A hyperdominant Xh chromosome may be defined as one which converts a Y chromosome in a zygote to an Xh chromosome by degrading it before replicating itself, by means of a self-regulating feedback loop akin to that of type 1 autosomal hyperdominance. Only a paired Xh or Xw chromosome can block this process. In a zygote formed by Xw Y and Xh Xw parents, the four possible outcomes are therefore: Xw Xh, Xw Xw, Xh Xh, and Y Xw. In a zygote formed by Xw Y and Xh Xh parents, the four possible outcomes are: 2 Xw Xh:2 Xh Xh.

Assuming a panmictic population in which all male-female crosses are equally likely, one may consider a population of 1 Xw Y:1 Xw Xw:1 Xh Xw:1 Xh Xh. The three equally likely crosses may be considered in the preceding paragraph. Summing the four possible outcomes of each of the three crosses, the ratio 3 Xw Y:3 Xw Xw:3 Xh Xw:3 Xh Xh, or the same parental ratio of the panmictic population. The parameters of the self-regulating feedback loop enabling total allosomal hyperdominance, in which the entirety of the allosome may be taken into consideration rather than only the homologous loci, may be adjusted to provide other sex ratios at a self-sustaining ratio in a panmictic population. Specifically, the likelihood with which the hyperdominant Xh converts Y to Xh, as well as the recognition interaction between Xh and Xw, may be used toward this end. Existing population parameters such as pre-existing sex ratio and differential rates of survival and reproduction may also be taken into account. The self-sustaining 3:1 ratio may be used as an example with respect to an ideal panmictic population.

Thus, the allosomal hyperdominance component of the invention teaches the means by which selected homologous genetic loci on sex chromosomes may exhibit hyperdominance under the category of partial allosomal hyperdominance. Also, the method of total allosomal hyperdominance teaches the means by which a sex ratio other than 1:1 may be self-sustaining in a panmictic population that recombines haploid genomes into new diploid zygotes through sexual reproduction. Furthermore, this is accomplished by means of chromosomal interaction and a self-regulating feedback loop in the zygote, which this invention teaches as a novel alternative to naturally occurring methods of segregation distortion in meiosis and gamete formation.

In any instance of hyperdominance, genetic material may be introduced to provide novel traits not currently or previously present to a large extent in a population. Horizontal gene transfer may be a known process to allow organisms to incorporate new genetic material not present in the genomes of their parental organisms. It may be in contrast to traditional methods of reproduction in which genetic material passes between ancestors and descendants by means of repeated parent to offspring transmission, known as vertical gene transfer. In the event that horizontal gene transfer precedes vertical gene transfer, the combined phenomenon can justly be referred to as diagonal gene transfer. In some embodiments hyperdominance may be used to provide a perennial lineage of sustained diagonal gene transfer in which the genetic material acquired from horizontal gene transfer may not be diluted by a preponderance of wild-type organisms.

### GLOSSARY

Complete dominance: as used herein complete dominance is the mode of inheritance in which the phenotype of a heterozygote is identical to that of the homozygote. One allele is sufficient such that providing a second copy of the allele provides no additional effect.
Incomplete dominance: as used herein, incomplete dominance refers to the mode of inheritance in which the phenotype of a heterozygote is distinct from that of the homozygote. The presence of a second copy of an allele leads to a different, observable result in the organism.
Engender: as used herein engender means to produce, cause, or give rise to or in some cases when used without an object engender means to be produced or caused; come into existence
Horizontal Gene Transfer: as used herein horizontal gene transfer means the process by which organisms acquire and transmit genetic material outside of reproduction
Vertical Gene Transfer: as used herein vertical gene transfer means the process by which organisms acquire genetic material from parental organisms and transmit it to their offspring
Diagonal Gene Transfer: as used herein Diagonal Gene Transfer means the process by which genetic material introduced by means of horizontal gene transfer is later propagated through vertical gene transfer.
Hyperdominant Founder: as used herein hyperdominant founder means an organism whose genetic construct achieves successful genetic penetrance in all future progeny regardless of repeated crosses with wild-type organisms in all successive generations.
Hyperdominance: as used herein hyperdominance means the mode of inheritance by which a trait achieves persistent, inter-generational penetrance regardless of the preponderance of crosses with wild-types in the monophyletic lineage of all descendants of a founder organism who all bear a genetic construct engendering said trait.
Perennial: as used herein perennial means enduring, perpetual.
Perpetual: as used herein perpetual means lasting for an indefinitely long time.

### BRIEF DESCRIPTION OF THE DRAWINGS

The accompanying drawings, that are incorporated in and constitute a part of this specification, illustrate several embodiments of the invention and, together with the description, serve to explain the principles of the invention:

FIG. 1 illustrates a diagram of genotypes involved in an episomal hyperdominance cross.

FIG. 2A illustrates a diagram of some embodiments of the invention with a lineage of inherited genotypes following from a cross between a founder organism and a wild-type organism.

FIG. 2B illustrates a diagram of some embodiments of the invention with a lineage of inherited genotypes following from a cross between a founder organism and a wild-type organism, with respect to a hyperdominant region of an autosome in type 2a autosomal hyperdominance.

FIG. 2C illustrates a diagram of some embodiments of the invention with an autosome pair as seen in type 1 or type 2a autosomal hyperdominance.

FIG. 3A illustrates exemplary models of lineage of inherited genotypes according to some embodiments of the inventive art herein.

FIG. 3B illustrates exemplary models of lineage of inherited genotypes according to some embodiments of the inventive art herein.

FIG. 3C illustrates exemplary models of lineage of inherited genotypes according to some embodiments of the inventive art herein.

FIG. 3D illustrates exemplary models of lineage of inherited genotypes according to some embodiments of the inventive art herein.

FIG. 3E illustrates exemplary models of lineage of inherited genotypes according to some embodiments of the inventive art herein.

FIG. 3F illustrates exemplary models of lineage of inherited genotypes according to some embodiments of the inventive art herein.

### DETAILED DESCRIPTION OF THE INVENTION

According to the present invention a perennial lineage may be engendered with hyperdominant traits that express consistent penetrance in all descendants of a founder organism. A genetic construct containing one or more genetic elements encoding a self-regulating feedback loop generates a regulatory RNA, polypeptide, or other gene product at or below a trigger level of concentration in a zygote and indicates with respect to the concentration level whether one or two copies of a genetic construct conferring a hyperdominant trait exist in said zygote.

This may be provided for by identifying one or more origins of replication for which DNA replication can be specifically initiated without affecting the origins of replication elsewhere in the genome of the organism. Additionally, origins of replication which can be concurrently activated for the replication of DNA along with the other origins of replication in the genome of the organism may be considered as well for genome replication beyond the initial zygote stage of development. This may be provided for by identifying one or more existing genetic elements encoding a regulatory RNA, polypeptide, or other gene product which directs the initiation of DNA replication at a specific, recognized origin of replication. Furthermore, this may be provided for by identifying one or more existing genetic elements encoding a regulatory RNA, polypeptide, or other gene product which acts to inhibit the production or action of the aforementioned regulatory RNA, polypeptide, or other gene product initiating DNA replication in order to suppress a successive round of DNA replication. Specifically, said gene product would only accumulate enough to block the next round of replication after a first round of DNA replication increased the expression of said gene product, for which the genetic elements giving rise to said gene product reside on the genetic construct whose replication is regulated by this mechanism.

Combining the genetic elements previously mentioned with one or more genetic elements which engender at least one hyperdominant trait may be enabled by the following methods. First, following the identification of the component genetic elements from various sources, these genetic elements may be excised through restriction endonuclease digestion and ligated into vectors for further amplification and study. As needed, directed mutagenesis may enhance one or more genetic elements through alterations to DNA sequences which may be manifested in the improved specificity and action of the regulatory RNA, polypeptide, or other gene product which is encoded. The genetic construct may be provided for by the restriction endonuclease digest, mechanical shearing, or sonic fragmentation of a suitable vector or chromosome as well as the restriction endonuclease digest of vectors holding the identified genetic elements previously described that are capable of producing a self-regulating feedback loop which used in concert. The specificity of restriction endonuclease digestion as well as the availability of DNA sequencing in the case of less specific mechanisms for breaking DNA into fragments generates a set of DNA fragments with known functions. The process of providing for the genetic construct continues with a DNA ligation to unify these DNA fragments into single molecule which combines the functions of the described genetic elements together. Alternatively, the identification of the various component genetic elements needed for the genetic construct may be followed by the de novo synthesis of the entire sequence. In this case, the enhancement of one or more genetic elements to improve the specificity and action of one or more regulatory RNA, polypeptide, or other gene product may be accomplished by changing the information of the nucleotide sequence prior to chemical synthesis rather than through directed mutagenesis. In either case, various genetic elements which together give rise to the self-regulating feedback loop and the ensuing inheritance pattern of hyperdominance may be combined to provide a novel genetic construct through the existing tools of recombinant DNA engineering.

A guided replication of a region within or the entirety of said genetic construct may produce two copies of the region within said genetic construct in the resultant homozygote, regardless of whether one or both parent gametes contained said genetic construct. In some embodiments a zygote may be converted from hemizygous to homozygous.

Some embodiments may include converting a zygote from heterozygous to homozygous. Gametes of an organism may be generated derived from the zygote, all of which may include one copy of the genetic construct.

Embodiments may also include conferring progenitorship upon a founder organism by means of first introducing said genetic construct into its genome. A recreation of the hyperdominant trait may be conferred by the genetic construct across successive generations with fidelity and consistent penetrance that define a perennial lineage.

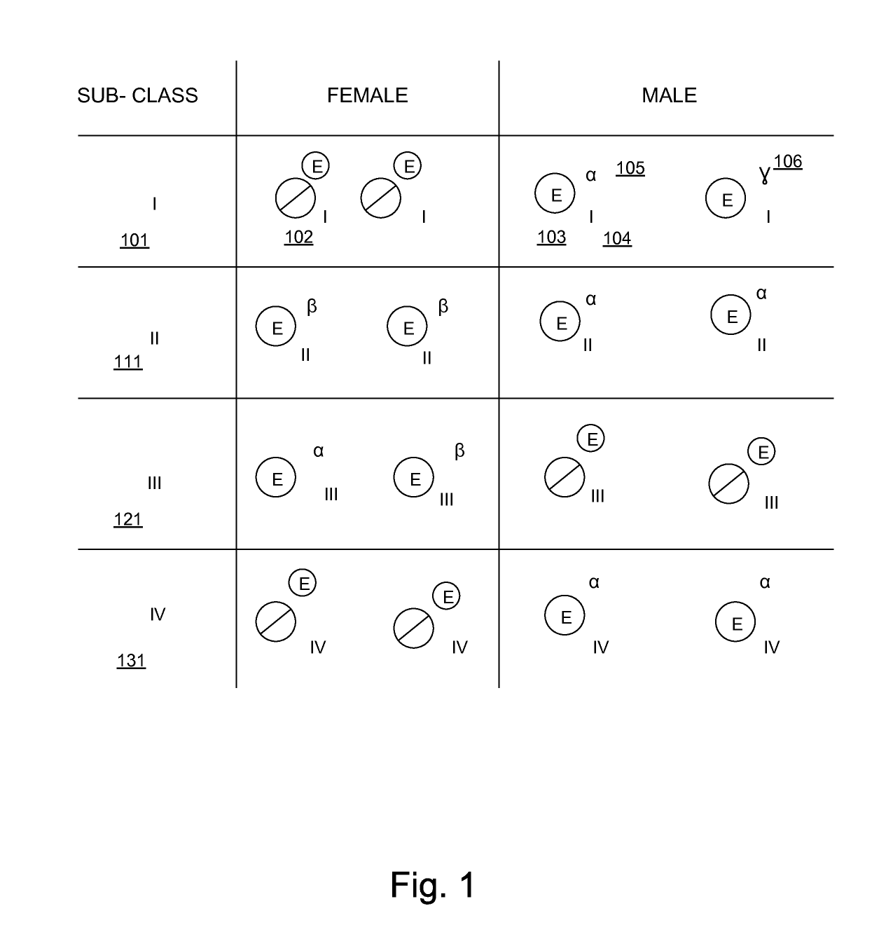

FIG. 1 in accordance with a preferred embodiment of the invention depicts genotypes involved in an episomal hyperdominance cross. Specifically, a series of different episomal hyperdominance subclasses are shown.

The table has five rows and three columns. The female organism, as shown in the second row and second column, has no genetic constructs 102 corresponding to the trait associated with subclass I 101 of the episomal hyperdominant genetic constructs, a genotype that could be described as wild-type respective to subclass I 101. The empty set symbol is augmented with an (E) for episomal and an “I” subscript for subclass I 101 to illustrate this genotype 102.

It should be noted that the Greek letters 105, 106 describing particular episomal hyperdominant genetic constructs such as 103, 104, and 105 refer specifically to variations within a particular subclass 101. As such, a single Greek letter 105 used across various subclasses 101, 111, 121, 131 may be assigned to multiple constructs while retaining its specific meaning within a particular subclass 101. In the system of notation pictured, this is denoted unambiguously by the subscript 104 which is used to identify the particular subclass 101 for a genetic construct 103, 104, 105 or the lack of a construct 102 for a particular subclass 101.

The male organism, as shown in the second row and third column, has two genetic constructs corresponding to the trait associated with subclass I 101 of the episomal hyperdominant genetic constructs. For the two episomal genetic constructs depicted in this part of the table, 103 denotes the episomal nature of this genetic construct. It is not part of any autosomal or allosomal chromosome in the organism's genome. The I symbol 104 denotes that this genetic construct is part of mutually compatible episomal hyperdominant subclass I 101, wherein multiple genetic constructs engendering the same hyperdominant trait offer genetic diversity within a subclass. The alpha 105 denotes which particular variant of the genetic construct within mutually compatible episomal hyperdominant subclass I this haploid partial genotype corresponds to. The gamma 106 similarly denotes which particular variant of the genetic construct for subclass I that the other haploid partial genotype corresponds to. Hence, the genotype of the male organism is alpha/gamma 105, 106 with respect to subclass I 101.

It is evident that episomal genetic construct replication for subclass I 101 did not take place in the male organism at the zygote stage, because the presence of two different 105, 106 constructs with respect to mutually compatible episomal hyperdominant subclass I 101 could not arise from a single construct's duplication. By the previously described function of the feedback loop, specifically the instance of said loop for mutually compatible subclass I 101, the non-replication of the episomal genetic construct for subclass I 101 necessarily implies that each parent gamete that produced the zygote stage of the male organism supplied its own haploid partial genotype for subclass I 101. Hence, both parents bore the hyperdominant trait engendered by the episomal genetic constructs within mutually compatible subclass I 101. One can then infer that the two parental genotypes of the male organism were either homozygous alpha 105 within subclass I 101 and homozygous gamma 106 within subclass I 101, or the same genotype as the male organism 105, 106 for one or both parents.

By application of the hyperdominant mode of inheritance, a cross between the organisms whose genotypes are described by this table may produce the following results. As the female organism is wild-type with respect to subclass I 101 owing to the absence 102 of any hyperdominant episomal genetic construct in said subclass, this parent may contribute no genetic construct of the mutually compatible subclass I 101. The male parent, in providing a haploid genome via gamete to the progeny of the cross, may contribute a single copy of either of the two variants 105, 106 of the episomal hyperdominant construct for subclass I 101. By the previously described function of the feedback loop, this may result in either homozygous alpha 105 or homozygous gamma 106 as the genotype with respect to subclass I 101 in the progeny.

For the cross of genotypes with respect to subclass II 111, it is apparent that the female parent can only contribute the beta variant of the genetic construct in mutually compatible subclass II 111, while the male parent can only contribute the alpha variant. Hence, the progeny of the cross may have genotypes with respect to subclass II 111 which are alpha/beta, as the feedback loop skips the replication process due to the presence of two constructs of the same subclass 111 in the zygote.

For the cross of genotypes with respect to subclass III 121, it is apparent that the female parent can contribute either the alpha or beta variants of the genetic construct in mutually compatible subclass III 121, while the male parent contributes no genetic construct for subclass III 121. Hence, the progeny of the cross may have genotypes with respect to subclass III 121 which are either homozygous alpha or homozygous beta, following the replication process coordinated by the previously described feedback loop.

For the cross of genotypes with respect to subclass IV 131, it is apparent that the female parent contributes no genetic construct for subclass IV 131. The male parent can only contribute a single copy of the alpha variant in mutually compatible subclass IV 131. Hence, the progeny of the cross may have genotypes with respect to subclass IV 131 which are homozygous alpha, following the replication process coordinated by the previously described feedback loop.

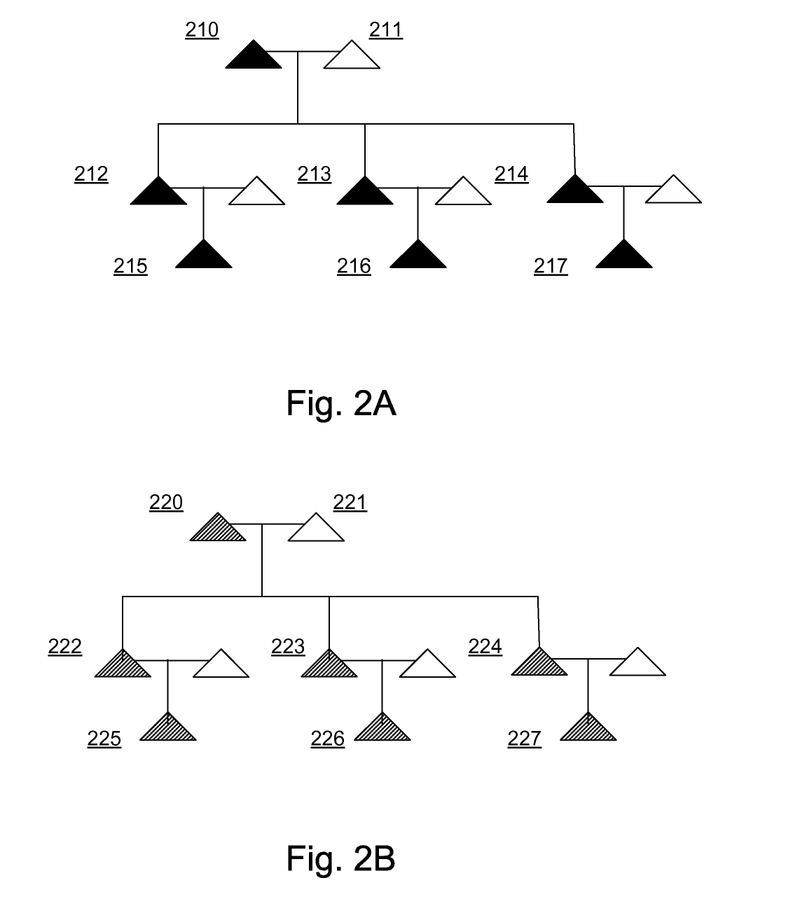

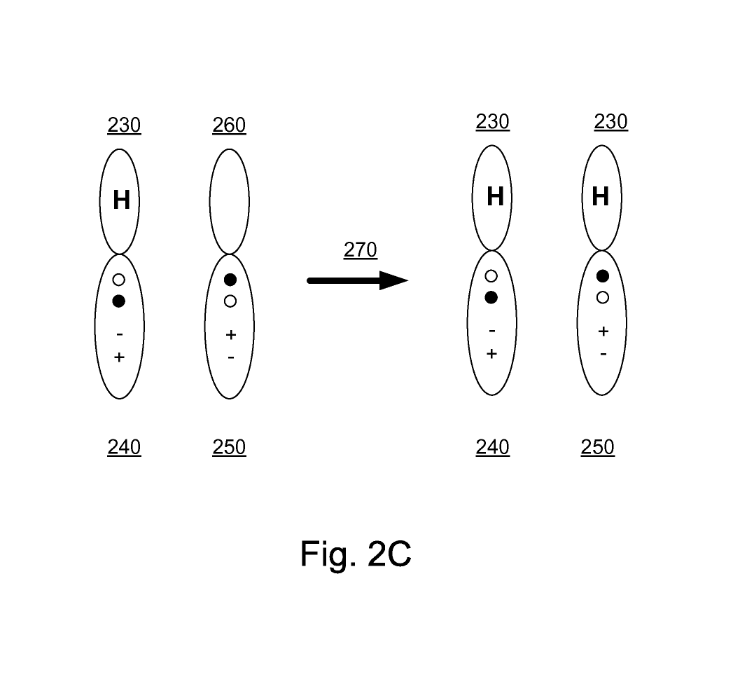

FIG. 2A in accordance with a preferred embodiment of the invention depicts a lineage of inherited genotypes following from a cross between a founder organism and a wild-type organism, with respect to the entirety of an autosome pair in type 1 autosomal hyperdominance.

FIG. 2B in accordance with a preferred embodiment of the invention depicts a lineage of inherited genotypes following from a cross between a founder organism and a wild-type organism, with respect to a hyperdominant region of an autosome in type 2a autosomal hyperdominance.

FIG. 2C in accordance with a preferred embodiment of the invention depicts an autosome pair as seen in type 1 or type 2a autosomal hyperdominance as well as the process employed specifically in type 2a autosomal hyperdominance.

For simplicity, the sex of organisms in the genetic family history is omitted. Females and males are equally represented as triangles, as only autosomal inheritance is considered here.

In the first parental generation of the family tree, the organism 210 may be a hyperdominant founder organism, and the organism 211 is a wild-type organism. A particular autosomal chromosome 230, 240, specifically a hyperdominant one, may be shown as it may be transmitted in a gamete from a given hyperdominant organism 210. The region 230 may be the hyperdominant region “H” of said autosome, and the remainder region 240 corresponds to remaining alleles on said autosome. For comparison, a particular wild-type homolog 250, 260 of said hyperdominant autosome 230, 240 may be shown as it may be transmitted in a gamete from a wild-type organism 211.

Under type 1 autosomal hyperdominance, the black shaded triangle genotype of the founder organism 210 corresponds to an entire hyperdominant autosomal chromosome 230, 240 which replicates to replace the wild-type autosomal chromosome 250, 260 in its entirety. In type 1 hyperdominance, existing genetic loci elsewhere on the hyperdominant autosome 240 outside the hyperdominant region “H” 230 replace all genetic diversity 250 in the wild-type autosomal chromosome 250, 260 along with the wild-type genetic region 260 corresponding to the hyperdominant region 230 of the hyperdominant homolog 230, 240.

Thus, the entire autosomal hyperdominant chromosome 230, 240 may be inherited in the first filial generation descendants 212, 213, 214 of the hyperdominant founder organism 210 and the wild-type organism 211 as an autosome pair consisting of two copies of the type 1 autosomal hyperdominant chromosome 230, 240. By the genetic inheritance mechanism of type 1 autosomal hyperdominance, the organisms 212, 213, 214 carry the same autosome pair, two copies of 230, 240, as the hyperdominant founder organism 210. Consequently, a cross of the first filial generation 212, 213, 214 with wild-types lacking the type 1 hyperdominant autosome 230, 240 in the same manner as the organism 211 did produces the second filial generation 215, 216, 217 that also carries the same autosome pair consisting of two copies of 230, 240.

The black shaded triangles 210, 212, 213, 214, 215, 216, 217 represent the inheritance of the type 1 hyperdominant autosome 230, 240 through the first 212, 213, 214 and second 215, 216, 217 filial generations following the parental generation of the founder organism 210 and the wild-type organism 211 with respect to type 1 hyperdominance, in a process that can be repeated through a perennial lineage.

Under type 2a autosomal hyperdominance, the basis for the family tree of FIG. 2B, the light shaded triangle genotype of the founder organism 220 corresponds to only the hyperdominant region “H” 230 of an autosomal chromosome 230, 240, where said region's replication replaces only the wild-type 250, 260 autosomal chromosome's corresponding region 260. In type 2a hyperdominance, the process of homologous recombination and homologous chromosome DNA template repair via dsDNA breaks 270 as controlled by the previously described feedback loop only takes notice of the number of hyperdominant regions in determining whether this replication and selective replacement may be to proceed. In this way, existing genetic loci 240 elsewhere on the type 2a hyperdominant autosome 230, 240 outside the hyperdominant region 230 do not replace the genetic diversity “as is” 250 in the wild-type autosomal chromosome 250, 260. The wild-type genetic region 260 corresponding to the hyperdominant region “H” 230 of the hyperdominant homolog 230, 240 may be the only region replaced.

Thus, the hyperdominant region 230 may be inherited in the first filial generation descendants 222, 223, 224 of the parental generation's hyperdominant founder organism 220 and the wild-type organism 221 within an autosome pair 230, 240, 250, to the right of process 270 in FIG. 2C, that has two copies of the hyperdominant region 230. By the genetic inheritance mechanism of type 2a autosomal hyperdominance, the organisms 222, 223, 224 carry two copies of 230 as the hyperdominant founder organism 220 did. Consequently, a cross of the first filial generation 222, 223, 224 with wild-types lacking the type 2a hyperdominant region 230 in the same manner as the organism 221 did produces the second filial generation 225, 226, 227 that also carries two copies of the type 2a hyperdominant region 230 on the type 2a hyperdominant autosome pair 230, 240, 250, even while preserving genetic diversity carried by wild-types on their existing genetic loci 250.

The light shaded triangles 220, 222, 223, 224, 225, 226, 227 may represent the inheritance of the type 2a hyperdominant region “H” 230 through the first 222, 223, 224 and second 225, 226, 227 filial generations following the parental generation of the founder organism 220 and the wild-type organism 221 with respect to type 2a hyperdominance, in a process that can be repeated through a perennial lineage.

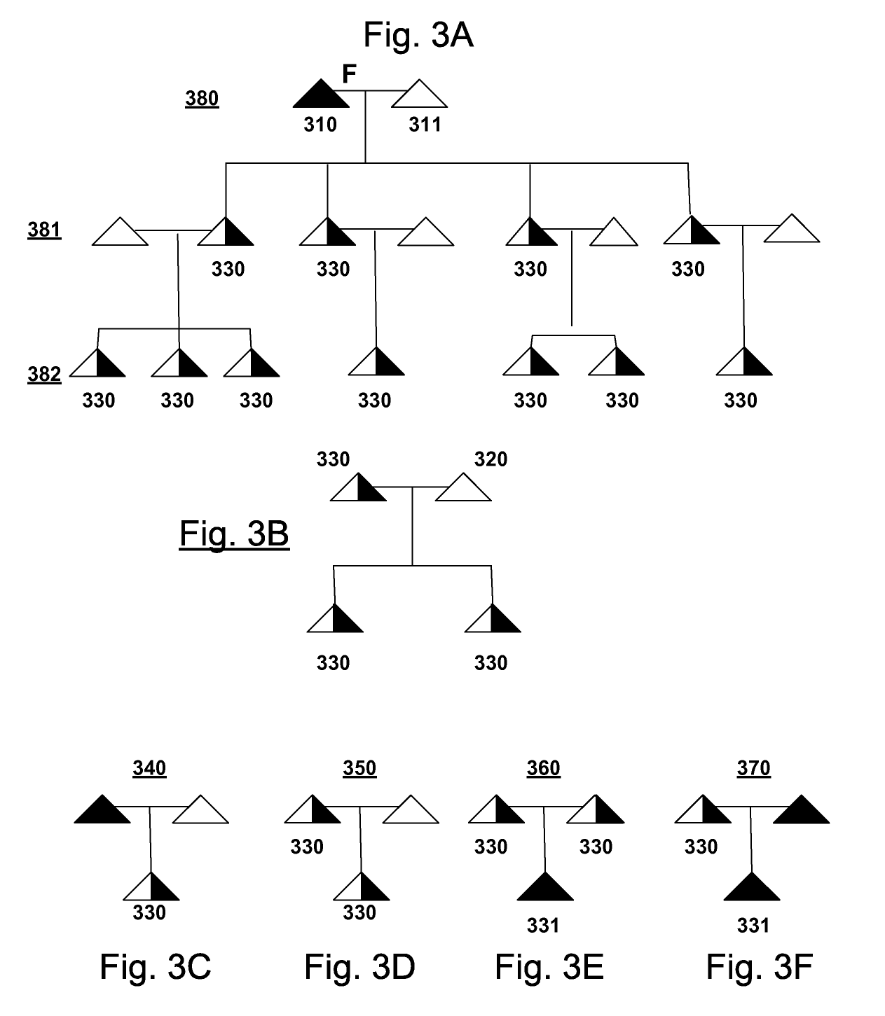

FIG. 3A in accordance with a preferred embodiment of the invention may depict a lineage of inherited genotypes following from a cross between a founder organism and a wild-type organism, with respect to type 2b autosomal hyperdominance, which allows for an intermediate genotype in the context of complete dominance as well as an additional intermediate phenotype in the case of incomplete dominance.

FIG. 3B in accordance with a preferred embodiment of the invention may depict a cross between the intermediate genotype of type 2b autosomal hyperdominance and a wild-type, which may allow for the continued undiluted inheritance of the hyperdominant trait.

FIG. 3C in accordance with a preferred embodiment of the invention may depict a cross between the homozygous genotype for the type 2b autosomal hyperdominant region and a wild-type, which yields the intermediate genotype of type 2b autosomal hyperdominance.

FIG. 3D in accordance with a preferred embodiment of the invention may depict a cross between the intermediate genotype for the type 2b autosomal hyperdominant region and a wild-type, which yields the intermediate genotype of type 2b autosomal hyperdominance.

FIG. 3E in accordance with a preferred embodiment of the invention may depict a cross between two organisms of the intermediate genotype of type 2b autosomal hyperdominance, which may yield the homozygous genotype for the type 2b autosomal hyperdominant region.

FIG. 3F in accordance with a preferred embodiment of the invention may depict a cross between the intermediate genotype of type 2b autosomal hyperdominance and the homozygous genotype for the type 2b autosomal hyperdominant region, which may yield the homozygous genotype for the type 2b autosomal hyperdominant region.

A founder hyperdominant organism 310 may be crossed with a wild-type organism 311, and these two organisms constituting the first parental generation 380. The superscript “F” describing the genotype of the founder hyperdominant organism 310 may be used to emphasize that the founder hyperdominant organism 310 has a distinct genotype with respect to the hyperdominant region when compared to descendant hyperdominant organisms 330 of the first 381 and second 382 filial generations in the perennial lineage following crosses with wild-types using the first parental 380 and first filial 381 generations, respectively. This may be specific meaning described by the superscript “F” on the founder hyperdominant organism 310 was not the part of the modes of inheritance particular to the previously addressed type 1 and type 2a of autosomal hyperdominance.

The half shaded triangle 330 referencing the members of the first 381 and second 382 filial generations in the perennial lineage denotes the intermediate genotype in which only one copy of the portion of the hyperdominant region engendering the hyperdominant trait may be present following the heterozygous to homozygous conversion of at least the portion of the hyperdominant region encoding the self-regulating feedback loop and potentially additional genetic loci with proximity to the region copied over. This may be in contrast to the fully shaded triangle 310, 331 which denotes the presence of two copies of the portion of the hyperdominant region engendering the hyperdominant trait, a consequence of having two complete hyperdominant regions, also known as the homozygous genotype for the type 2b autosomal hyperdominant region. This fully shaded triangle genotype 310, 331 can arise from initially conferring progenitorship upon a hyperdominant founder organism 310, from a cross 360 between two organisms of the intermediate half shaded triangle genotype 330, from a cross 370 between the intermediate half shaded triangle genotype 330 and the fully shaded triangle genotype 310, 331, or from a cross, not shown, between two organisms of the fully shaded triangle genotype 310, 331.

The intermediate half shaded triangle genotype 330, depicted in a way to reflect heterozygosity, refers to a heterozygous hyperdominant region, namely with respect to the portion of the hyperdominant region which engenders the hyperdominant trait. In the zygote stage of an organism with the intermediate half shaded triangle genotype 330, homozygosity may be enforced elsewhere on the concurrent autosome by means of the processes regulated through the feedback loop, such that the elements encoding the feedback loop constitute the minimal genetic region undergoing the heterozygous to homozygous transition. Any cross 350 between this intermediate genotype 330 and a wild-type 320 results in the same intermediate genotype 330 once again, such that it may be referred to as the intermediate genotype of type 2b autosomal hyperdominance. This was previously examined in the cross between the family tree's first filial generation 381 and wild-types 311 to produce the second filial generation 382, indicating the persistence of the hyperdominant trait via the undiluted heritability of the intermediate genotype 330 across generations 380, 381, 382.

In a cross 340 between a homozygous genotype for the type 2b autosomal hyperdominant region 310, 331 and a wild-type 311, the intermediate genotype 330 of type 2b autosomal hyperdominance may be observed in progeny. Since the inheritance pattern of this cross 340 occurs regardless of whether the parent having the homozygous genotype 310, 331 for the type 2b autosomal hyperdominant region may be a hyperdominant founder organism 310 or not 331, the superscript “F” may be omitted.

In a cross between the intermediate genotype 330 and another organism of the same intermediate genotype 330, the progeny genotype pattern of 360 may be observed. This pattern 360 shows the emergence of an organism which may be homozygous 331 for the hyperdominant region even under the type 2b autosomal hyperdominance model that allows for heterozygosity of the hyperdominant region. In a cross 370 between the intermediate genotype 330 and a homozygous genotype 310, 331 for the type 2b autosomal hyperdominant region, the homozygous genotype for the type 2b autosomal hyperdominant region may be observed in progeny 331.

In this way, in some embodiments the heterozygosity 330 of the hyperdominant region allowed for in type 2b autosomal hyperdominance persists over generations 381, 382 and can lead to the hyperdominant region becoming fixed by the following process. First, wild-types decrease in relative abundance over generations as seen in the 340, 350 crosses. Secondly, type 2b autosomal hyperdominant organisms which are heterozygous 330 for the hyperdominant region cross 360 with each other to yield progeny of the homozygous genotype 331 for the type 2b autosomal hyperdominant region.

Thirdly, the cross 370 between organisms heterozygous 330 for the hyperdominant region and organisms homozygous 310, 331 for the hyperdominant region results in progeny homozygous 331 for the hyperdominant region. Over an arbitrarily large number of generations, this process gradually leads to the fixity of the hyperdominant region even with type 2b autosomal hyperdominance being employed, since genotypes other than the homozygous genotype 331 for the type 2b autosomal hyperdominant region eventually disappear from the population.

### CONCLUSION

A number of implementations have been described. Nevertheless, it will be understood that various modifications may be made without departing from the spirit and scope of the disclosure. For example, various forms of the flows shown above may be used, with steps reordered, added, or removed. Also, although several applications of persistent inheritance of hyperdominant traits have been described, it should be recognized that numerous other applications are contemplated. Accordingly, other implementations are within the scope of the following claims.

What is claimed is:

1) A method for engendering a perennial lineage with hyperdominant traits, the method comprising the step of providing a genetic construct containing one or more genetic elements encoding a self-regulating feedback loop.

2) The method of claim 1, wherein said feedback loop guides the replication of a region within or the entirety of said genetic construct.

3) The method of claim 2, wherein two copies of said region within said genetic construct convert said zygote from hemizygous to homozygous.

4) The method of claim 2, wherein two copies of said region within said genetic construct convert said zygote from heterozygous to homozygous.

5) The method of claim 2, wherein two copies of said genetic construct convert said zygote from hemizygous to homozygous.

6) The method of claim 2, wherein two copies of said genetic construct convert said zygote from heterozygous to homozygous.

7) The method of claim 2, wherein the organism derived from said zygote generates gametes, all of which include one copy of said genetic construct.

8) The method of claim 1, further comprising the conferring of progenitorship upon a founder organism by means of first introducing said genetic construct into its genome.

9) The method of claim 7, wherein the hyperdominant trait conferred by said genetic construct is recreated across successive generations with fidelity and consistent penetrance that define a perennial lineage.

10) The method of claim 9, engendering a perennial lineage with hyperdominant traits that express consistent penetrance in all descendants of said founder organism.

11) The product(s) of claim 8, wherein said hyperdominant founder organisms are made progenitors by the methods taught by the invention.

12) The product(s) of claim 10, wherein organisms are descendants of a monophyletic lineage originating from a single hyperdominant founder organism.

13) The product(s) of claim 10, wherein organisms are descendants of multiple hyperdominant founder organisms.

14) The method of claim 1, wherein said genetic construct is derived by use of horizontal gene transfer.

15) The method of claim 14, wherein said genetic construct is part of a perennial lineage featuring vertical gene transfer.

16) The method of claim 15, wherein horizontal and vertical gene transfer are combined in the process of diagonal gene transfer.

17) The product(s) of claim 16, wherein organisms acquire genetic material in the process of diagonal gene transfer.

18) A method for engendering a perennial lineage with hyperdominant traits, the method comprising the steps of:
providing a genetic construct containing one or more genetic elements encoding a self-regulating feedback loop, wherein the genetic construct generates one or more of a regulatory RNA, polypeptide, and other gene product, at a concentration level at or below a trigger level of concentration in a zygote, and wherein said concentration level indicates whether one or two copies of a genetic construct conferring a hyperdominant trait exist in said zygote;
providing replication of at least a region within said genetic construct guided by the action of the genetic construct;
producing two copies of the region within said genetic construct in the resultant homozygote;
generating gametes of the organism derived from said zygote, all of which include one copy of said genetic construct;
conferring progenitorship upon a founder organism by means of first introducing said genetic construct into its genome; and
replicating the hyperdominant trait conferred by the genetic construct across successive generations with fidelity and consistent penetrance that define a perennial lineage.
